(19) United States
(12) Patent Application Publication
(10) Pub. No.: US 2025/0057443 A1
(43) Pub. Date: Feb. 20, 2025

(54) A CONFORMABLE PRESSURE SENSOR PAD

(71) Applicant: VEINSENSE LTD, Wadhurst (GB)

(72) Inventors: Arnold HARPIN, Wadhurst (GB); Stephen BARKER, Shirley (GB)

(21) Appl. No.: 18/728,355

(22) PCT Filed: Dec. 6, 2022
(86) PCT No.: PCT/GB2022/053100

§ 371 Date: Jul. 11, 2024

(30) Foreign Application Priority Data
Jan. 12, 2022 (GB) 2200359.4

(51) Int. Cl. A61B 5/103; A61B 5/00; A61F 13/00; A61F 13/08

(52) U.S. Cl. CPC A61B 5/1036; A61B 5/6804; A61F 13/00051; A61F 13/08; A61F 2013/00957

(57) ABSTRACT

A conformable pressure sensor pad 109 comprising: a sensor arrangement 130 comprising a central column 10 of segments and outer columns 11 of segments, wherein the segments in the outermost columns are tapered outwardly, and wherein a pressure-sensitive sensor 22 is mounted in a plurality of the segments in the central column; and a conformable pad 110 including a plurality of pouches 62, the pouches 62 being in fluid communication with each other, each segment 10, 11 of the sensor arrangement 130 being positioned on a respective pouch 62, the plurality of pouches 62 being shaped to be positioned between a respective segment 10, 11 and the skin surface of a patient in use.

### BACKGROUND OF THE INVENTION

Venous leg ulcers are a life-changing and sometimes life-limiting medical condition that responds poorly to current, but long established treatments. This causes long-term distress and possible immobility to the patent, and the long-term nature of the treatment and limited effectiveness makes it an expensive condition to treat.

The key to effective treatment is to bandage (or compress by other means) the leg in such a way that there is a pressure gradient up the leg, starting with a higher pressure (typically 5.2 kPa or 40 mmHg) at the ankle, gradually reducing to 2.6 kPa (or 20 mmHg) at the top of the calf, just below the knee. (Other graduated compression profiles may have an effect.)

Conventional bandaging techniques attempt to achieve such a profile by wrapping a bandage at approximately uniform tension up the leg. The tension may be gauged by noting the amount of stretch for example, in circles pre-imprinted on the bandage. Theoretically, for a uniform tension, the pressure exerted by a bandage on a cylindrical structure (such as approximating a leg) is inversely proportional to the radius of curvature (based on Laplace's Law). This, and the ‘experience’ of the applier of the bandage, is used to produce what is hoped is the correct graduated compression profile. However, typically, the pressures exerted in this way are not as expected (being often higher) and worse, the profile is not uniform. This could make the condition worse as a high spot in the pressure up the leg would tend to inhibit the desired flow of blood and other fluids (such as lymph) up the leg, possibly even causing more serious conditions. In addition, the high pressure and non-uniformity of the pressure gradient may cause unnecessary patient discomfort which results in a low treatment uptake (concordance); patients tend to remove the bandage on comfort grounds or even re-fit themselves for comfort, thus completely invalidating the treatment.

Ideally a pressure sensor can be deployed, which is fitted between the leg and bandage (or compression garment, or wrap system) to measure the pressures at points up the leg (typically about 4 to 6). However, it is important that the pressure sensor itself does not cause a significantly erroneous pressure reading by perturbing the pressure where it sits. Typically, inserting an object between the leg and bandage causes a local (possibly harmful, and definitely a contributor to inaccuracy) elevation in pressure. Attempts to mitigate these difficulties have included making small, thin sensors, but these tend to be very expensive and often delicate in practical use, relying on such technologies as fibre gratings and complex interrogation schemes. An added difficulty is that the compressing pressures are quite low, and the sensor needs to be immune to shear and twisting effects.

WO2017/174984 discloses a sensor comprising first and second layers, separated by an elastic spacer. The separation of the first and second layers is determined by capacitive or optical means. This gives an indication of the pressure applied to the top and bottom layers. The sensor is essentially rectangular in cross section.

### SUMMARY OF THE INVENTION

According to a first aspect of the invention, there is provided a conformable pressure sensor pad comprising: a sensor strip comprising an elongate member that is segmented, the segments being defined by continuous longitudinal and transverse grooves, such that adjacent segments are hinged together, wherein the longitudinal grooves define a central column of segments and outer columns of segments, wherein the segments in the outermost columns are tapered outwardly, and wherein a pressure-sensitive sensor is mounted in a plurality of the segments in the central column; and a conformable pad including a plurality of pouches, the pouches being in fluid communication with each other, each sensor segment being positioned on a respective pouch, the plurality of pouches being shaped to be positioned between a respective sensor segment of the sensor strip and a surface of a patient in use.

It will be understood that the surface of the patient could be the skin of the patient (i.e. the pouches are in direct contact with the skin) or there could be an intermediate material between the skin of the patient and the pouches, e.g. a covering such as a fibrous padding layer or stockinette.

Preferably, the width of each respective pouch of the plurality of pouches is substantially equal to the width of the respective segment of the sensor strip. The width is the direction substantially of the segment and pouch tangent to the limb and perpendicular to the axis of the limb when the segment and pouch are in use. Matching the widths of the pouches to those of the segments ensures an even pressure distribution from the segments, through the pouches and onto the patient's limb.

Preferably, the adjacent pouches are in fluid communication with each other via a fluid communication means located at the ends of adjacent pouches. The ends of the pouches are the distal regions of the pouches along the axis of the limb when the pouches are in use. Fluid (i.e. air) from one pouch can be transferred into a neighbouring pouch through the fluid communication means. The interconnection means allow fluid and thereby pressure to be transferred between pouches and control the tendency of a vessel full of a fluid to adopt a spherical shape.

Preferably, the fluid communication means is located at alternative ends of each neighbouring pouch to create sets of interdigitated pouches. Interdigitated pouches provide the required flexibility to encircle the limb, while being easier to manufacture than separate pouches.

Preferably, a fluid communication means is located at the edges of the pads to create a set of interdigitated pouches. This alternate layout may provide improved fluid communication in some geometries, and may also be easier to manufacture.

Preferably, a fluid communication means is an interdigitated section running centrally through the pad which creates sets of interdigitated pouches. This alternate layout may provide improved fluid communication in some geometries, and may also be easier to manufacture.

Preferably, the fluid communication means comprise pipes and/or openings. The pipes extend between neighbouring pouches and interconnect the interior regions of those pouches. An opening between pouches can simply be considered a pipe of zero length. Pipes and openings advantageously allow greater relative twisting between neighbouring pouches than other kinds of intercommunication means.

Preferably, a pouch comprises fluid communication at opposing ends, and the interconnection means at each end provide fluid communication with different adjacent pouches.

It will be understood that any suitable combination of the above-described fluid communication means could be used in order to create the interdigitated pouches.

Preferably, each pouch is a pouch of fluid. The fluid may be a gel such as silicon gel, a liquid such as water, or a gas such as air or nitrogen. The pouch may be fully filled with the fluid or partially filled with that fluid and partially filled with another substance. For example, the pouch may be fully filled with a liquid, or partially filled with a liquid and partially with a gas. Filling each pouch with fluid helps provide the pad with greater stiffness and ensures a more uniform pressure distribution.

Preferably, each pouch is a bellows structure with crenelated side walls. A bellows structure provides a flat upper and lower surface, in which the upper and lower surfaces can translate and rotate relative to each other. This advantageously ensures that large contact areas are maintained between the upper pouch surface and the sensor strip, and between the lower pouch surface and the patient's skin, thereby ensuring low and uniform pressure distribution.

Preferably, each sensor segment extends along the length of a respective pouch. The length is the dimension substantially aligned with the axis of the limb. Extending the sensor segment along the length of its respective pouch maximises the contact area between the segment and pouch, improving the pressure distribution between the segment and pouch.

Preferably, the length of each sensor segment is substantially equal to the length of the respective pouch. This advantageously ensures that the pouch is separated from the bandage along its whole length by its respective sensor segment. Were the bandage to make contact with a pouch directly, a local pressure distortion could ensue.

Preferably, the back surface of the sensor strip comprises an adhesive or high friction coefficient material. The “back surface” of the sensor strip is the surface on the opposite side to the bandage, i.e. the surface in contact with the conformable pad. The adhesive or high friction coefficient material ensures that the segments of the sensor strip do not slide across the surface of the conformable pad, while still allowing each segment to twist or depress into the pouch below it.

Preferably, the conformable pad is pre-tensioned in a direction parallel to the circumference of the limb. The conformable pad may be pre-tensioned by slightly stretching the pad around the limb to generate tension, and then securing the edges of the pad to the limb under tension. This is followed by application of the bandage, which applies further tension. The applied pre-tension is low enough to not significantly affect the pressure sensor reading. Applying a pre-tension to the conformable pad increases the frictional forces generated between the conformable pad and limb, ensuring that the conformable pad is kept in place.

According to a second aspect of the invention there is provided a pressure sensor garment or bandage comprising a conformal pressure pad as described hereinabove integrated therein. A garment or bandage comprising a conformal pressure pad may be easier to fit or more comfortable to wear than a standalone conformal pressure pad.

Preferably, the pressure sensor garment further comprises a plurality of pressure sensor pads. The pressure pads can be positioned within the garment to only cover the required areas of the patients skin, without overly impeding the flexibility of the garment.

Preferably, each pressure pad is positioned relative to one another to be interspersed along the length of a patient's limb onto which the pressure sensor garment is applied. Interspersing the pads along the length of the limb aligns the pads with the pressure gradient to be created on the limb.

Preferably, each pressure pad is aligned with one another along the length of a patient's limb. “aligned” could for example refer to aligning the centroid of each pressure pad along the axis of the limb, or to aligning an exterior surface or surfaces of each pouch. Aligning the pressure pads facilitates easy positioning of the sensor segment on top of a plurality of pads, and further ensures that an accurate and even pressure gradient is created over the length of the patient's limb.

Preferably, two pressure pads in the plurality of pressure pads differ from each other in a physical parameter, the physical parameter being one of a list comprising the shape, size and stiffness of the pads. This allows pads to be configured for the geometry of the limb, and the pressure to be applied, in that particular location. For example, the width of a pad covering the calf portion of the leg may be increased to take account of the limb having a larger circumference in this region. In another example, a conformable pad positioned lower on the patient's limb may be made stiffer to reflect the higher required pressure towards the patient's ankle.

Preferably, the garment is configured to at least partly encircle a patient's limb. Full or partial encirclement of the limb ensures that the garment is retained on the limb and cannot easily fall off.

Preferably, the garment is one of a list comprising a sock, a wrap, a sleeve, and a band.

### DESCRIPTION OF THE FIGURES

Examples of the present invention will now be described in detail, with reference to the accompanying Figures, in which:

FIG. 1(a) shows a simplified plan view of an example of an elongate segmented sensor strip;

FIG. 1(b) shows a cross sectional view along the line A-A in FIG. 1(a);

FIG. 1(c) shows cross sectional view along the line B-B in FIG. 1(a) to illustrate the hinging and separation of the segments of the sensor strip in use;

FIG. 2 shows a simplified top view, side view and end partial view of an alternate example of an elongate segmented sensor strip;

FIG. 3 shows a cross sectional view along the lines A-A, B-B, and C-C in FIG. 2;

FIGS. 4(a)-(c) show different exemplary embodiments of a conformal pad as a rectangular slab for use with the elongated sensor strip shown in the previous Figures;

FIGS. 5(a) and (b) show the application of a bandage onto a limb over a conformable pressure sensor pad according to the invention, respectively with and without the presence of a layer between the pad and bandage.

FIGS. 6(a)-6(d) show plan and cross-sectional views of an example of a sensor strip;

FIG. 7 is a diagram showing the geometry of a segmented sensor strip;

FIG. 8 is a diagram showing pressure enhancement due to sensor height;

FIGS. 9(a)-9(d) show views of a sensor cell;

FIG. 10 shows a top for the sensor cell of FIG. 7;

FIG. 11 shows an exemplary sensor strip;

FIGS. 12(a), 12(b) and 12(c) illustrate fluid pouches for use with the sensor strip of FIG. 6;

FIG. 13 shows fluid pouches in combination with tapered sensor segments;

FIG. 14 shows a garment incorporating a plurality of conformable pressure pads;

FIGS. 15(a) and 15(b) are diagrams illustrating the geometry of the bandage and segments.

FIGS. 16(a), 16(b) and 16(c) are graphs showing the results of an experimental trial using an embodiment of the invention.

### DESCRIPTION OF THE INVENTION

In the present disclosure, a medical dressing (or more generally “dressing”) should be understood to include any dressing, bandage (such as a stretch bandage), hosiery (such as elasticated hosiery), or a wrap-based system that may be applied to a patient in a relevant medical setting (especially compression dressings). The medical dressing may be in direct contact with a patient's skin, or there may be intermediate layers/dressings between the medical dressing and the patient's skins. The term “bandage” used herein should be understood to refer more generally to such a medical dressing.

In preferred examples, we measure the displacement of a flexible elongate sensor strip using optical proximity sensors mounted to the strip. Capacitive sensors may also be used. Such a sensor strip is shaped in such a way that it does not significantly alter the pressure exerted by a bandage, in that the sensor strip conforms to the curvature of the leg where it is fitted. In addition, the sensor strip is tapered to avoid any pressure enhancement at the edge of the sensor strip.

In a preferred example, the sensor strip is from 300 to 450 mm long (typically 380 mm long) and 10-15 mm wide. By virtue of the tapering structure and the segmentation to follow the radius of curvature, the sensor strip can be typically 2-4 mm thick, which allows cost-effective sensing techniques (the sensor strip may have a relatively short service life, typically days or weeks per patient).

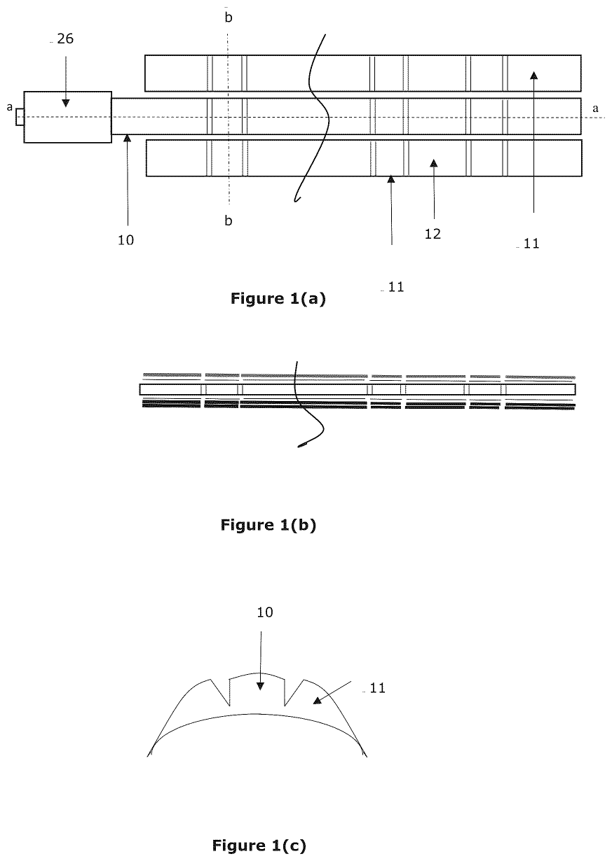

Referring now to the Figures, a sensor strip of the type shown in FIG. 1 can be used by mounting it along a calf with an electrical connector 26 placed just below the knee in typical operation.

The segmentation of the sensor strip allows it to follow the curvature of the leg at any point. The sensor strip comprises a central column 10 and outer columns 11 and each of the segments 12 may have a rigid top and bottom surface.

In preferred examples, the widths of the segments 12 are typically 10 mm, e.g., not more than 20 mm, and as low as 5 mm. The thickness of the sensor strip should be as low as practically possible, but 1-5 mm is a typical workable range.

In some examples, there is one outer column 11 either side of the central column 10 as shown in FIG. 1, but it is possible and may enhance performance if there are two outer columns 11 on each side of central column 10—i.e., a total of 5 segments. Typically, the length of each segment is around 50 mm. This may be dictated by the compliance of the sensor strip and leg geometry and might be as low as 10 mm. 50-60 mm is often the largest practical size.

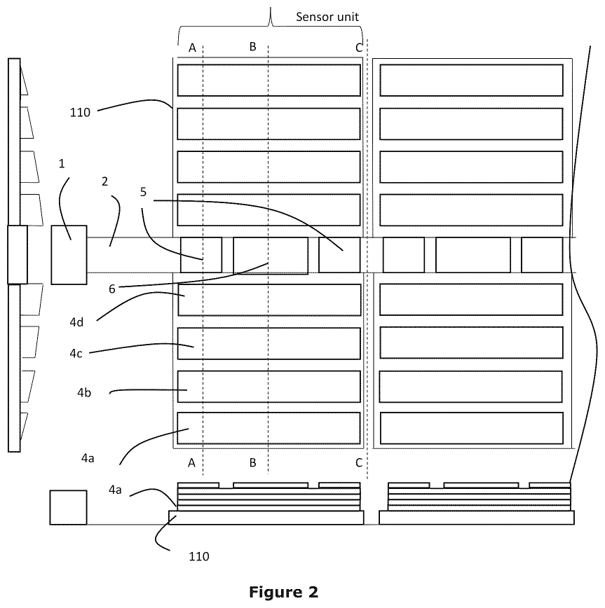

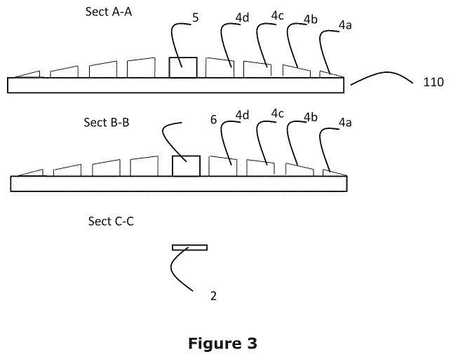

Referring to FIGS. 2 and 3, the segmented sensor strip comprises a connector 1 for connecting the sensor strip to an external power supply/control unit (not shown). The connector may be located at a distal end of a central column 2 which supports the electronic components of the sensor strip. The column 2 may comprise an elongate flexible PCB strip. Alternatively or additionally, the column 2 may comprise flexible elements that act as hinges along its length to increase its flexibility. Pressure sensor elements 6 are located along the length of the column 2. The pressure sensors are interspersed by spacers 5. The spacers 5 are the same height as their neighbouring pressure sensor elements 6, so as to provide a continuous profile for the bandage to follow and thereby avoid irregularities in the bandage path which could cause pressure distortions.

Arranged parallel to axis of the central column 2 are tapered segments 4a, b, c, d. The tapered fillets ensure that the bandage path maintains a large radius of curvature while passing over the sensor strip, thereby preventing undesirable local pressure enhancements around or on top of the sensor strip. The height of the inner edge of the innermost segment 4d may be equal to the height of the pressure sensors and spacers 5 on the central column 2. The outermost segment 4a may be tapered to zero height at its outermost edge. In an embodiment, the spacers 5 and/or tapered segments 4 may be formed of silicone, in order to provide the requisite flexibility and deformability.

The undersides of the central column 2 and tapered segments 4 rest against conformable pads 110. In the embodiment shown in FIG. 2, there comprises a plurality conformable pads 110 in the form of airbags. The conformable pads 110 are arranged along the length of the central column 2. Using a plurality of conformable pads provides increased flexibility along the axis of the central column. In an embodiment, the conformable pads 110 may differ from each other in properties such as size, shape or stiffness, in order to for example better suit different parts of the limb. In an alternate embodiment, there may comprise a single conformable pad 110 which extends along the entire length of the central column 2. The or each conformable pad 110 includes a plurality of pouches (not shown in FIGS. 2 and 3 for clarity purposes), and the central column 2 and each tapered segment 4a-d of the sensor arrangement is positioned on a respective pouch of a conformable pad 110. The central column 2 and each tapered segment 4 may be directly attached or joined to a different pouch of the conformable pad 110 as shown, allowing the sensor strip to flex around the axis of the central column 2. In an alternative embodiment (not shown), the segments 4 and central column 2 may be hinged together along their elongate edges to form a single flexible sensor strip component. This component then rests or is attached to a conformable pad or pads 110.

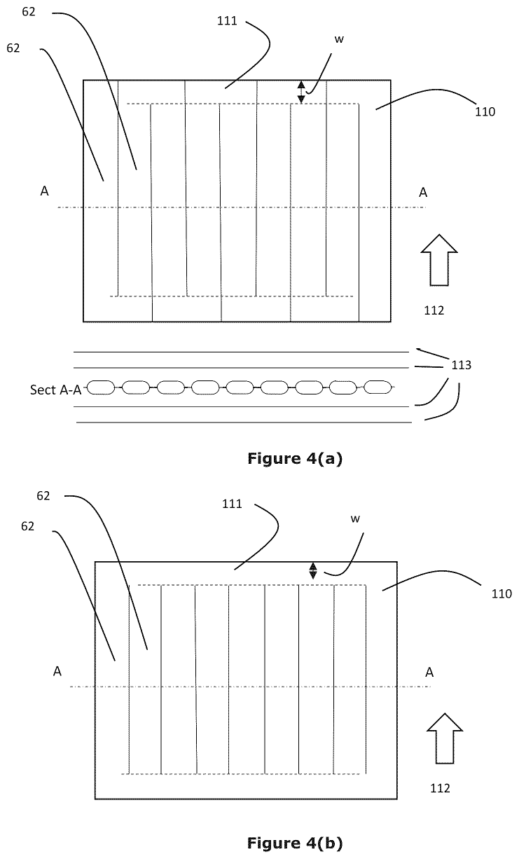

FIG. 4(a) shows a conformable pad 110 to be used in combination with any of the embodiments of sensor strips as described above, so as to form a conformable pressure sensor pad according to the invention. The conformable pad 110 is in the form of an airbag (i.e. pouches filled with air). The airbag 110 is shown in plan view and in a flat configuration—in use, the airbag would be placed against a wearer's limb. The airbag 110 is segmented as shown with interdigitated segments 62, joined through interdigitated sections 111 at alternate ends. The pattern of segments 62 and interdigitated sections 111 in the airbag 110 shown FIG. 4(a) creates a “zig-zag” shaped airbag 110. In other words, the fluid flow through the conformable pad 110 flows along the length of a segment 62 in one direction and then along the length of the neighbouring segment 62 in the opposite direction, and so on. In this way, the interdigitated sections 111 act as interconnection means between the segments 62, which allow the airbag 110 to be bent around a limb with less resistance. The axis of the limb is shown by the arrow 112. By contrast, an unsegmented airbag filled with air would try to straighten out when bent, which would apply excess pressure partly due to volume change. The interdigitated sections 111 have a width w, as indicated in FIG. 4(a). The width w should be sufficiently large to allow fast fluid flow between segments 62 and from one end of the airbag 110 to the other. Alternatively or additionally, the interdigitated sections 111 should be reinforced to ensure they stay open. If w is too narrow or the interdigitated sections 111 collapse, then the airbag 110 will not re-adjust.

FIG. 4(a) also shows a cross-section side view through the airbag 110 along the line A-A, with layers 113 of low-friction material above and below the airbag 110. The layers 113 are not shown in the other views of FIG. 4. It has been found that in use that the airbag 110 will, as it is pressurised to different profiles, tend to extend or contract in length. It is important this movement is not translated to a shearing force between a limb and the airbag 110, as any obstruction of the airbag's 110 movement may cause erroneous pressure readings. To prevent this, layers 113 of film may be inserted around the airbag 110 on the upper and lower sides of the airbag 110. The upper layers 113 sit between the airbag 110 and sensor strip or bandage. The lower layers 113 sit between the airbag 110 and the limb. The layers 113 should be as thin as possible in order to mitigate local pressure enhancement, while being sufficiently stiff and tough to prevent stretching or tearing when in use. Each layer 113 may comprise a material with a low friction material, preferably PTFE or other plastic film. As shown in FIG. 4(a), there may comprise two or more stacked layers 113 of film on one or both sides of the airbag 110. The surface of any of the airbag 110, limb, bandage or sensor may be rough or sticky, introducing shear forces which resist relative movement. Using two layers 113 ensures that a low-friction interface exists at least at the contact surfaces between the layers 113, allowing the layers 113 to shift independently. The friction is defined by the inner surfaces of the two 113 layers.

FIGS. 4(b) and 4(c) show alternate configurations for the airbag 110. In FIG. 4(b), the airbag 110 comprises an interdigitated section 111 which runs along the edges of the airbag 110. In FIG. 4(c), the interdigitated section 111 runs centrally across the airbag 110. In each case, the interdigitated section serves to provide fluid communication means between the segments 62. In alternate embodiments, an airbag 110 could comprise a combination of the interdigitated sections 111 shown in each of FIGS. 4(a), (b) and (c).

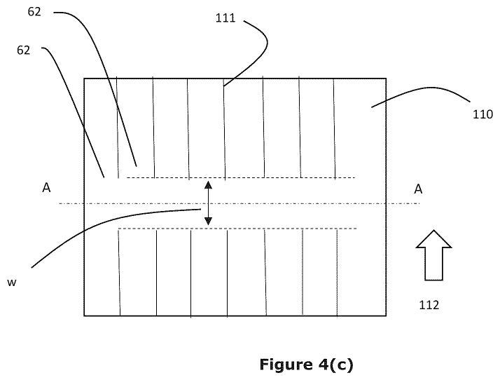

FIGS. 5(a) and 5(b) show the effect of providing low-friction layers 113 on the upper and/or lower sides of the airbag, as shown in the cross section side view of FIG. 4(a). In FIG. 5(a), no low-friction layers are provided. As the bandage 14 is wound onto the limb 13 (in this case counter clockwise, as shown by the arrow) the right most (leading edge) of the airbag 110 will be overly compressed. This will give incorrect pressure correction. The airbag 110 will then try to adopt the optimal profile, which is shown in FIG. 5(b). Without the low-friction layers 113, the illustrated bandage section would have to stretch as the airbag 110 reshapes itself, thereby increasing its tension and altering the pressure. However, if there are layers 113 (not shown in FIGS. 5(a) and 5(b) for clarity purposes) as previously described between the airbag 110 and bandage 14, the airbag 110 is free to readjust without stretching the bandage 14 to a greater extent.

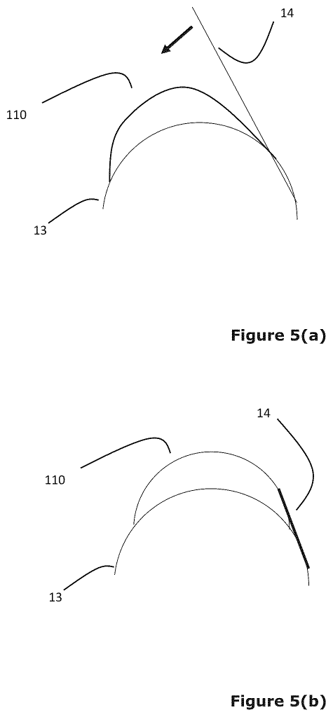

FIG. 6(a) shows a sensor strip formed by mounting a rubber strip 20 on a flexible PCB substrate 21. FIG. 6(b) is a section A-A along FIG. 6(a), which shows in more detail a connector 26, optical proximity sensors 22, each formed in cavity 23, with an (optional) stiffener 24 and reflectors 25. FIG. 6(c) is a section along B-B in FIG. 6(a). The position of each cavity 23 is illustrated in FIG. 6(d).

Preferably, the upper surface of the rubber strip 20 is textured, or a layer of a suitable material is laid on top, in order to enhance contact reproducibility.

In some examples, a sensor strip comprises a one-piece rubber strip or similar structure with a grooved surface to provide the segmentation, below which is attached a flexible PCB to route the signals to the electrical connector and to mount the optical proximity detectors.

FIG. 6(b) shows the flexible PCB 21 running the length of the sensor strip. The sensor cavity 23 is a recess, preferably a circular recess, formed in the rubber strip 20, the internal top surface of which may be coated with suitable reflective material 25, preferably a diffuse Lambertian reflector such as a suitable white paint printed onto the rubber.

In FIG. 6(d), the top surface of each sensor cavity is shown as square, but circular is an option. A square may be used as it presents a uniform edge for the bandage to land on, given the curved geometry the sensor strip is designed to be used on e.g., a human leg.

In an embodiment, the contact area with the bandage should be constant and preferably as close to 100% of the apparent contact area as possible. Black, Closed-cell, Firm Grade Neoprene Foam is a suitable material. The spring constant of the sensor strip and therefore the full-scale excursion of the sensor strip is determined largely by the Youngs Modulus of the rubber foam (HT800), but depends on the area of the recess 23. Optionally, voids could be moulded into the rubber around the sensor cavity to further reduce the area of rubber between the top and bottom of the sensor strip and hence the effective Youngs Modulus of the rubber.

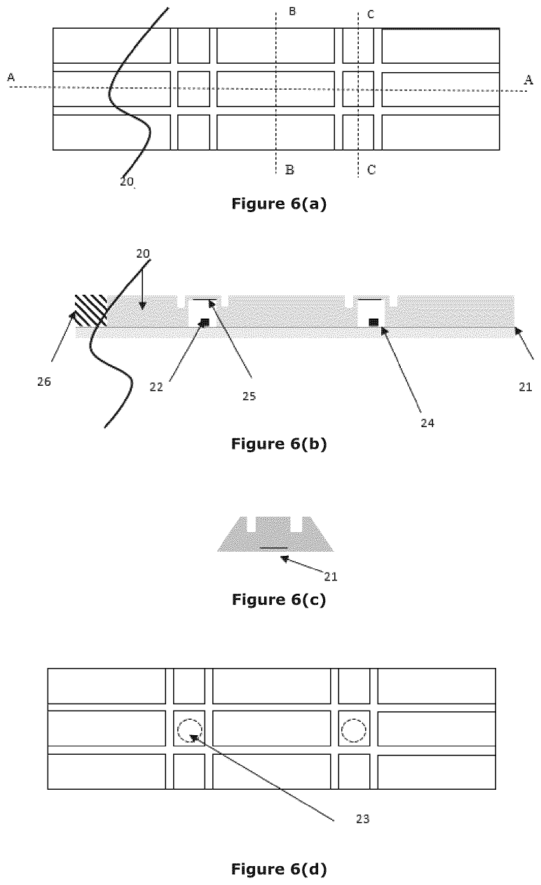

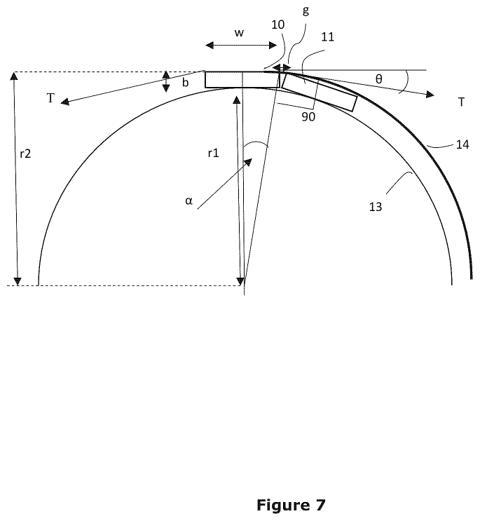

FIG. 7 shows how the sensor strip geometry works. For clarity, only one outer column 11 is shown to the right of the central column 10, as opposed to a sensor strip construction where there is an outer column either side. The drawing shows in addition a mandrel 13 and a bandage 14.

It can be shown that the pressure exerted on the flat upper surface of the sensor's central column 10 will be the same as if the sensor strip was not there (other than a small change of a few percent due to the fact that the sensor thickness of typically 3 mm adds to the effective radius of the leg which might be 35 mm or more), if the bandage leaves the edge of the central column 10 at the correct angle θ, where this angle is the angle of the tangent to the curvature of the bandage shown by the bold curved line. The force exerted on the sensor strip and hence the leg is given by the equation F=T sin θ. Where F is the downward force and T is the tension in the bandage.

The central column 10 has a width of w, and a thickness b. The mandrel 13 can be considered as a rigid cylinder with radius r₁. The bandage path is shown by the arrow with a tension T. The bandage on the left hand side is not shown. It is assumed that the bottom of the central column 10 is in complete contact with the mandrel 13. As will be discussed below, complete contact can be achieved by means of a conformal pressure sensor pad positioned between the columns 10, 11 and the mandrel 13. The angle α is given by

$$α = {tan}^{- 1} \frac{w}{2 {r}_{1}}$$

If the bandage path were to follow the gradient of the mandrel 13 as it comes off the central column 10, then the pressure applied by this central column 10 can be calculated using the equation:

$$P = \frac{T sin α}{2 w}$$

This equation can be simplified by noting that:

$$α = {sin}^{- 1} \frac{w}{2 \sqrt{( {{r}_{1}}^{2} + {( \frac{w}{2} )}^{2} )}}$$

$$So$$

$$P = \frac{T}{\sqrt{( {{r}_{1}}^{2} + {( \frac{w}{2} )}^{2} )}}$$

As w tends to 0, this tends to the value expected by Laplace's equation

$$P = \frac{T}{r}$$

For values of w of 12 mm, at a radius n in the range 35 mm to 55 mm, the error in the pressure measurement P is in the range 1.2% down to 0.5%. Therefore if the bandage 14 can be arranged to follow the gradient of the mandrel 13 the correct pressure will be measured.

It will be appreciated that if the outer column 11 were not there the sensor strip would read a greatly exaggerated pressure, and this pressure would be exerted locally on the leg, as can be seen from the comparative example in FIG. 7. The pressure could be increased by as much as a factor of 5—a significant and potentially harmful increase. Using the equation for the pressure exerted against the central column P=2T sin θ, the pressure enhancement can be calculated for exemplary values of the parameters. These are shown in Table 1 below:

TABLE 1

| r1 | w | b | α | Θ | Pressure |
|---|---|---|---|---|---|
| (mm) | (mm) | (mm) | (deg) | (deg) | enhancement factor |
| 35 | 12 | 6 | 9.7° | 40° | 3.8 |
| 35 | 12 | 1 | 9.7° | 26° | 2.6 |
| 55 | 12 | 6 | 6.2° | 32° | 4.8 |
| 55 | 12 | 1 | 6.2° | 19° | 2.9 |

The pivot and segmentation need to ensure the bandage follows to within a certain accuracy the same path as it would if the sensor strip was a continuous (unsegmented) flexible layer, as opposed to rigid segments joined together. It can be shown geometrically and theoretically that as long as the gap between segments is small (<1 mm) and the column width is <15 mm that the error in pressure is <10%. Similar considerations apply to the design of a one-piece segmented structure.

The segmentation allows the use of a relatively rigid material to form the sensor strip of only a few mm in thickness, which enables cost-effective mass manufacturable sensor strips.

Referring again to FIG. 7, it will be appreciated that while the bandage 14 passes onto segments 10, 11 of equal width and height the pressure will be correct. However, the same local pressure enhancement will occur when the bandage 14 eventually leaves the outermost segment and moves onto the mandrel 13. Furthermore, there is no guarantee that the pressure sensors in the sensor strip are reading the correct pressure as is elsewhere under the bandage. In other words, there may still be an undesirable local pressure enhancement in a location not measured by a pressure sensor.

For example, consider an example where the gap g between neighbouring segments is 2 mm, the radius r₁ of the mandrel 13 is 55 mm, and the width w of the central column is 12 mm. As above, the half width angle α would be 6.2°. The gap g between neighbouring segments 10 and 11 can be considered approximately horizontal. The drop of the bandage 14 between the neighbouring edges of segments 10 and 11 should therefore be 0.2 mm. Thus, if the segment 10 is taller than segment 11 by just 0.2 mm, the drop of the bandage would double and the local pressure would double. Since the local pressure enhancement is very sensitive to the relative heights of neighbouring segments, the local pressure enhancement between segments may be greater than what is measured directly underneath the segments by the pressure sensors.

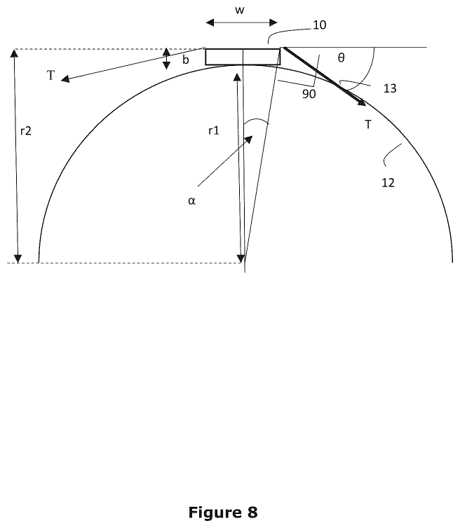

FIGS. 9(a) to 9(c) show an example sensor cell 6 of a pressure sensor that can be used in the sensor strip of the invention, e.g. the sensor strip from FIG. 2, in more detail. FIGS. 9(a) and 9(b) are respectively top and side views of the sensor showing how it is mounted on a flexible strip 2 (typically 12 mm width). FIGS. 9(c) and 9(d) are respectively top and section views of sensor 6 without a top element fitted, showing pressure equalisation levers, 61, and spring, 60.

FIG. 9(c) shows a top view of the sensor with the top element, upon which the bandage acts, removed. Three levers are used to ensure that regardless of the pressure distribution on the top pf the sensor, the same force will be applied to the spring 60. The levers are hinged to the base of the sensor, 6, so they can communicate the force, applied to the pins along the lever (roughly half way), 63 to the spring.

FIG. 9(d) shows a section EE through FIG. 9(c), showing how the three levers all bear down on the same point of the spring. The spring is preferably made of stainless steel. It is shown as having cut outs, but could be a continuous diaphragm, which would also have the advantage of preventing the ingress of dust and other contaminants to the sensor cell. The section EE also shows the reflector 64, and the optical proximity sensor. The reflector ideally has a Lambertian bottom surface to ensure consistent reflection of the optical signal back to the proximity sensor. As the distance between the reflector and the proximity sensor changes due to applied load to the spring, via the top of the sensor, the power reflected back to the proximity sensor gives an electrical signal proportional to the distance and hence the applied pressure.

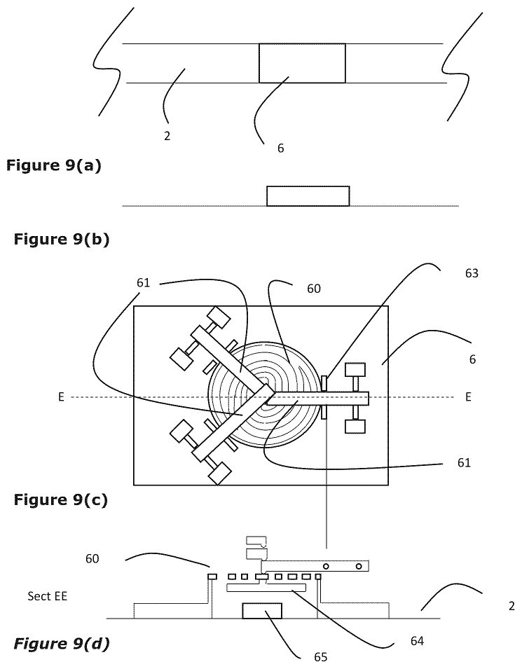

FIG. 10 shows the top element 7 of the complete sensor 6 which sits on top of the levers 61, and one set of lugs 71 that are a push fit onto the pin 63 that passes through a lever. Only one of three lugs is shown.

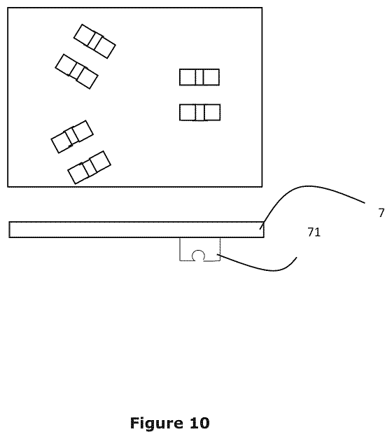

An exemplary sensor strip 50 is shown in FIG. 11, which may be one of the sensor strips described earlier. In this example, segment pads 51 of each segment of the sensor strip 50 are smaller than rigid base sections 52 on which the segment pads 51 rest (the segment pads on the left side of the illustration have been omitted for clarity). It is not necessary for the segment pads 51 to be as wide as the base sections 52 because the bandage 53 will still follow the correct path when the segment pads 51 have a smaller area than the bases 52, and a smaller contact area will help with contact area issues. If the segment pads 51 are too wide compared to the taper angle (i.e. the reduction in height of successive pads) the bandage 53 will not contact properly. For example, the pressure peak if a sensor is 12 mm wide and 4 mm high with no tapering would be four times the anticipated pressure of a bandage on a radius of 35 mm, and four and a half times for a radius of 55 mm. Further examples of the local pressure enhancement were given in Table 1 above.

If the tapering between segment pads 51 is gradual enough, the angle of the bandage 53 is only increased slightly at each segment pad 51 and the pressure elevation is within acceptable limits (e.g. a tapering width of around 50 mm will result in a pressure increase of around 10%).

The inventors have found that this pressure elevation can be further mitigated by positioning a conformable pad in under the sensor strip in the form of pouches being in fluid communication with each other. In this arrangement, each segment/flap 61 of the sensor strip is provided with a corresponding fluid-filled pouch 62 (alternatively referred to as a bladder or sac or fluid layer) as shown in FIG. 11(a). The fluid may comprise a gel, a liquid such as water, or a gas such as air. The pouch may be fully filled with the fluid or partially filled with that fluid and partially filled with another substance. For example, the pouch may be fully filled with a liquid, or partially filled with a liquid and partially with a gas. The fluid may be at ambient pressure, or else it may be pressurised above or below ambient pressure. The pouches 62 in FIG. 11(a) are shown schematically and are intended to be in fluid communication with one another.

When using a continuous layer of fluid, the fluid is preferably contained within a sealed pouch (e.g. a rubber bag) with thin walls (i.e. of negligible thickness compared to/at least one order of magnitude thinner than the thickness of the fluid layer) that are have a compliance large enough so as exert non-negligible pressure when bent round the limb. This can be achieved using a fluid sandwiched between two layers of cured silicone rubber, although other materials could also be used.

The fluid will act to remove the pressure highlights, although in the case of a gel it will not completely equalize the pressure everywhere. However, when a bandage or dressing is applied over the sensor strip 50, the segments will arrange themselves to give a uniform pressure without highlights even at the edge. This can be visualized as the outer segment, subjected to a higher force at the outer edge, will tend to tip, which will result in an equilibrium when the angle of the bandage is reduced to the point where the force on the outer segment balances the restoring forces from the fluid. There will be a pressure elevation under the segments, but this will be uniform and of the order of 10% if the tapering is sufficient.

Using a segmented pad with interconnected individual fluid pouches under each flap acts to remove the unevenness due to the flat segments pushing on a curved surface, and this arrangement also removes the requirement of the rubber container to be so elastic.

To allow for the pressure to be equalised, the fluid-filled pouches 62 are all in fluid communication with each other (e.g. interconnected by small pipes or openings between adjacent pouches 62, or means of interdigitated sections, as will be discussed below). The fluid communication means are not shown in FIG. 11(a). In this case, the tendency of a vessel full of air to adopt a spherical shape can be controlled compared to using a single continuous bladder or pouch, and each pouch 62 does not need to stretch excessively as it is bent or deformed round the mandrel or limb 63. This allows a completely even pressure distribution to be applied to the limb 63. The edge effect will be eliminated by the tilting of the outer taper as described earlier, and all segments will adopt the position required (which will tend to be circular). Preferably, the width of each pouch 62 may equal to the width of the segment/flap 61 under which the pouch 62 is positioned.

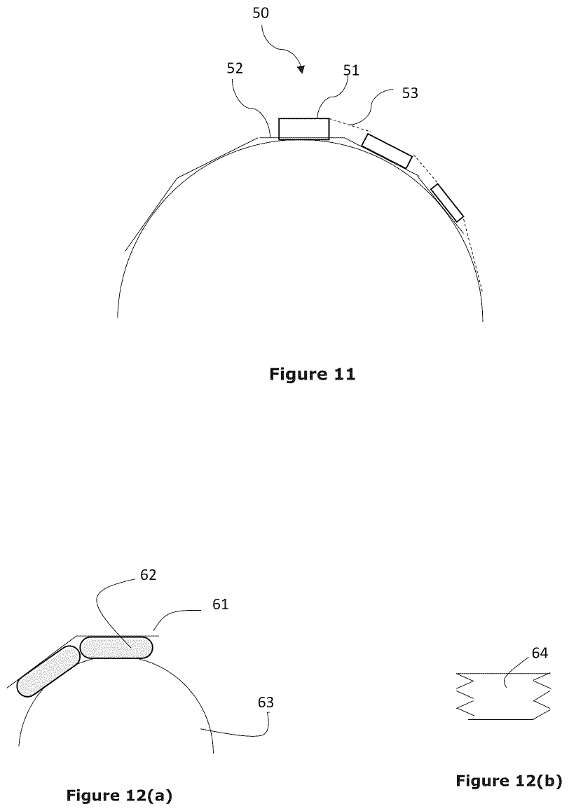

The pouches 62 may be pouches of fluid as shown in FIG. 12(a), or they may alternatively be interconnected bellows type structures 64 as shown in FIG. 12(b). FIG. 12(c) shows interconnected bellows type structures 64 arranged on a mandrel 63 in the manner of the fluid filled pouches 62 of FIG. 4. Unlike FIG. 12(a), the segments/flaps 61 are not shown. The fluid communication means between bellows structures 64 are also not shown.

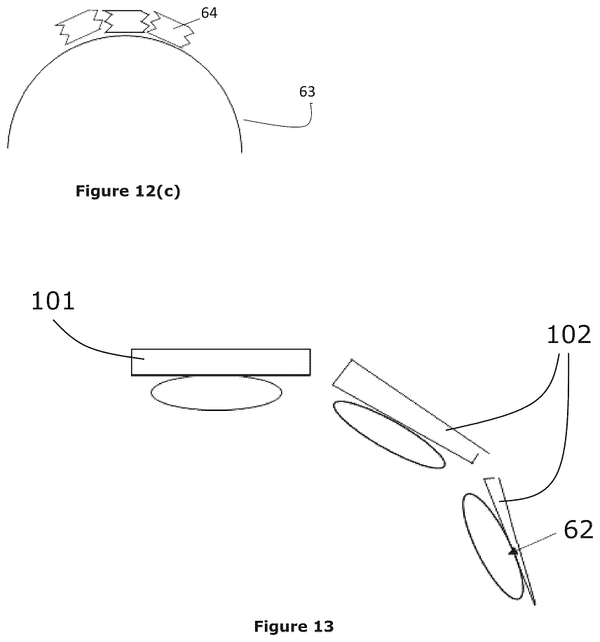

Referring to FIG. 13, it can be seen that each of the segments 62 has on top either the central sensor column 101 or a side column 102 comprising a tapered fillet (segment) 102 of rubber. In an embodiment, each segment 62 is 12 mm wide. Although in theory the segments 62 should adjust themselves, it is advantageous that the segments are relatively unperturbed as they must all present roughly equal contact area top and bottom this be equal between segments 62. There may also be other effects that mean the pressure might not be accurately transferred. As the segments 62 are compressed differently, the stiffness of the material they are made of and effects such as the hammock effect will affect their operation. The hammock effect is the effect of an inflated bag to only let something sink into it a certain amount. FIG. 13 is a schematic diagram which does not show the fluid communication between the pouches for clarity purposes.

The conformal pressure pad and tapered fillets work in combination to mitigate these effects and reduce local pressure distortions. As has been previously discussed, small errors in the dimensions or positions of segments can lead to local pressure distortions. This problem also exists for tapered segments. However, if the tapered segments are dimensioned and positioned so as to keep local pressure enhancement below a reasonable level (say no more than ˜10% fluctuation), then the conformal pressure pad will be able to distribute the uneven load ensure a uniform pressure is applied. Furthermore, as previously discussed the edge effects between segments will be mitigated by the tilting and/or translation of the segments on the pouches. The pouches can accommodate repositioning of segments by up to 1 mm, which is sufficient to accommodate height differences between segments that arise from manufacturing tolerances. Repositioning of the segments also enables different radii of limb to be accommodated, giving optimally uniform pressure distribution and optimally accurate pressure readings across a range of patients regardless of the size of their limb. In an embodiment, the taper profile of the segments 101, 102 is optimised for a smaller limb radius and the airbags 110 perform adjustment to accommodate a larger limb radius.

In the above arrangements, the thickness of the fluid layer is preferably chosen to be thick enough to accommodate the tipping of the outer flaps, which will generally be 1-3 mm.

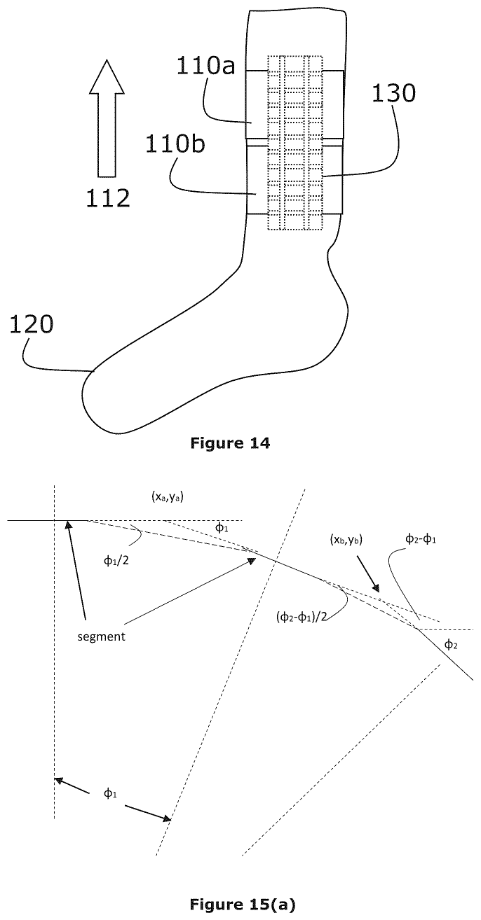

FIG. 14 shows a garment 120 in the form of a sock to be worn on a patient's foot and leg. A sensor strip 130 is shown in position over the garment 120, with dashed lines to indicate that the sensor strip 130 does not form part of the garment 120. The sensor strip 130 is positioned parallel to the arrow 112, which indicates the axis of the limb. The garment 120 includes a plurality of conformable pads 110a, 110b, located in the limb-covering section of the garment 120. Each conformable pad 110a, 110b comprises a plurality of pouches as previously described, although the pouches are not shown in FIG. 14. The conformable pads could be attached to the outer surface of the garment 120, attached to the inner surface, or integrally formed within the garment 120. The conformable pads 110 may fully encircle the limb, or alternatively they may only extend across a certain portion of the limb's circumference.

The conformable pads 110a, 110b are positioned between the sensor strip 130 and the limb (not shown), and aligned along the axis of the limb 112. The conformable pads 110a, 110b may be optimised for their particular position on the patient's limb. Consider that the upper conformable pad 110a is to be positioned further up the patient's leg than the lower conformable pad 110b. Physical parameters of the upper conformable pad 110a may be increased relative to the lower pad 110b to account for its positioning further up the limb. For example, the width of the pouches may be increased and/or the number of pouches in the pad may be increased to take account of the limb having a larger circumference over the calf portion of the leg. The conformable pads 110a, 110b may also or instead have different stiffness profiles in different regions of the limb to ensure the optimal pressure gradient is applied. For example, the lower conformable pad 110b may be made stiffer to reflect the higher required pressure towards the patient's ankle. The stiffness profile of the conformable pads 110a, 110b can be altered by for example using a thicker skin construction, using a different pouch geometry such as bellows, or using a different fluid within the pouches or a fluid at a different pressure or viscosity.

Although the sensor strip 130 in FIG. 14 is shown as a single sensor strip, in other embodiments there may be a plurality of individual sensor strips, each of which would be associated with a corresponding individual conformable pad.

Alternatively, rather than comprising a plurality of conformable pads, the garment 120 could comprise a single elongate conformable pressure sensor pad which extends along the axis of the limb 113 for a desired length.

The garment 120 could be a longer sock which extends above the knee. In alternative embodiments, the conformable pads 110a, 110b are integrated into another kind of garment such as a sleeve or band, so that the garment has an open end rather than an end which covers the patient's hand or foot. The conformable pressure pads could also be integrated into a wrap which is wrapped around the patient's limb or covers part of the limb. To facilitate application and removal and to ensure an optimum fit, a loosening/tightening means such as a row of buttons or a zip running down the side of the garment 120 may be incorporated.

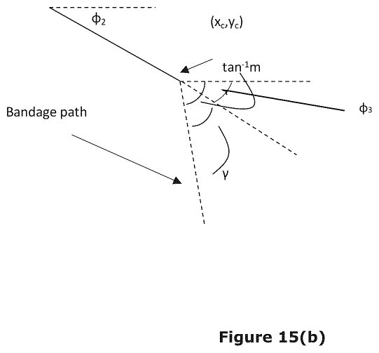

An explanation of how the ideal taper profile can be numerically calculated is provided with reference to FIGS. 15(a) and (b). In FIG. 15(a), the horizontal line labelled ‘segment’ is the top of the sensor and the two solid lines to the right are the tops of adjacent segments. It will be appreciated that it has no effect on the calculation if the segments are so close that they touch. (The gaps are there to allow the dotted construction lines and angles to be clear on the diagram). We assume, without justification, that they dispose themselves symmetrically as shown in FIG. 15(a). Being mounted on a fluid means, the segments are free to adjust their tilt and height. Referring to FIG. 15(b), the items labelled “segments” are flat stiff regions disposed on a bag of fluid—where we ignore the effects due to the gaps between the sensors. The point of this abstraction is that the pressure under each segment (henceforth we will call it a flap) is equal. Then further imagine that the right-most flap has the bandage run off onto the limb, as shown in FIG. 15(b). The following equations then apply:

$${φ}_{1} = {φ}_{2} - {φ}_{1}$$

We can be rearranged to:
Similarly, φ₂=2φ₁
and φ₃=3φ₁

### Finally,

$$γ = {tan}^{- 1} m - {φ}_{2}$$

$$γ = {φ}_{1} / 2$$

These equations can be solved numerically and we can show that the pressure enhancement is as stated.

We can then obtain a solution that gives a pressure enhancement of order 10% for four segments and a 6 mm high sensor. The fact that this solution is stable, in that each segment is static, shows that this is a viable way for the segments to arrange themselves. For lower height of sensor the results will be lower. It is worth noting that the angle the bandage leaves the mandrel at ‘programmes’ the angles of the remaining segments, or the angle is shared amongst the number of segments.

### Experimental Data

A trial was performed to demonstrate the effectiveness of the conformal pressure pad system. 6 volunteers (identified below as patients 101-106) were selected for the application of a graduated-tension bandage to be applied to a leg over the conformal pressure pad. The pressure pad comprised 4 pressure sensors arranged in line along the axis of the leg. The bandages were applied by two nurses with many years experience applying bandages in the conventional manner. 3 tests (identified below as tests 1-3) were conducted on each patient, each test with a different bandage system. Test 1 used the Actico® bandage system, test 2 the Coban™ bandage system and test 3 the Urgo™ K2 bandage system. In each case, the aim was to apply the bandage with a pressure graduation starting at 40 mmHg at the ankle, and progressively lowering to 20 mmHg just below the knee.

In tests 1 and 2, the bandage was applied over the conformal pressure pad without the nurses being allowed to see the readings from the pressure sensors. In other words, the nurses had to rely on the conventional “by-eye” methodology for generating the graduation profile. In test 3, the nurses applied the bandages while referencing the pressure sensor readings, which were displayed to them. The sensor readings were recorded by an independent observer once bandaging was finished. The sensor readings were then analysed to determine whether a graduated profile had been successfully applied. An average pressure reading for each sensor was also calculated across the 6 patients.

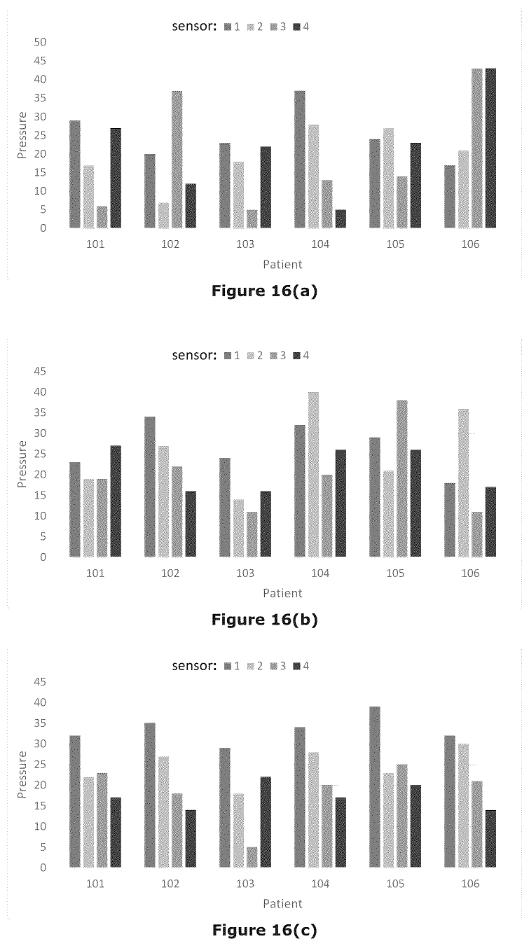

Tables 2-4 below show the results obtained from tests 1-3. The results from tests 1-3 are also shown in graphic form in FIGS. 16(a)-(c) respectively. As shown by tables 2 and 3, in the majority of cases even skilled and experienced nurses were unable to generate an accurate pressure gradient. A pressure gradient was successfully applied only to patient 104 in test 1, and patient 102 in test 2, i.e. on only 17% of the total applications. By contrast, Table 4 shows that when the nurses were able to view the pressure sensor readings while applying the bandages, they were able to produce a consistent and accurate pressure sensor reading almost every time, with 83% of applications being successful. The unsuccessful pressure gradient application on patient 102 in test 3 was only due to a hardware failure in the conformal pressure pad used in that instance. These tests demonstrate the consistent inaccuracy of the conventional method, and the consistent improvement in bandage pressure gradients which can be obtained by using this invention.

TABLE 2
Actico ® bandage

| | Patient | | | | | | |
|---|---|---|---|---|---|---|---|
| Sensor | 101 | 102 | 103 | 104 | 105 | 106 | Average |
| 1 (closest to | 29 | 20 | 23 | 37 | 24 | 17 | 25 |
| ankle) | | | | | | | |
| 2 | 17 | 7 | 18 | 28 | 27 | 21 | 20 |
| 3 | 6 | 37 | 5 | 13 | 14 | 43 | 20 |
| 4 (closest to | 27 | 12 | 22 | 5 | 23 | 43 | 22 |
| knee) | | | | | | | |
| Graduated | No | No | No | Yes | No | No | 16.60% |

TABLE 3
Coban ™ bandage

| | Patient | | | | | | |
|---|---|---|---|---|---|---|---|
| Sensor | 101 | 102 | 103 | 104 | 105 | 106 | Average |
| 1 (closest to | 23 | 34 | 24 | 32 | 29 | 18 | 27 |
| ankle) | | | | | | | |
| 2 | 19 | 27 | 14 | 40 | 21 | 36 | 26 |
| 3 | 19 | 22 | 11 | 20 | 38 | 11 | 20 |
| 4 (closest to | 27 | 16 | 16 | 26 | 26 | 17 | 21 |
| knee) | | | | | | | |
| Graduated | No | Yes | No | No | No | No | 16.60% |

TABLE 4
UrgoK2 ™ bandage

| | Patient | | | | | | |
|---|---|---|---|---|---|---|---|
| Sensor | 101 | 102 | 103 | 104 | 105 | 106 | Average |
| 1 (closest to | 32 | 35 | 29 | 34 | 39 | 32 | 34 |
| ankle) | | | | | | | |
| 2 | 22 | 27 | 18 | 28 | 23 | 30 | 25 |
| 3 | 23 | 18 | 5 | 20 | 25 | 21 | 19 |
| 4 (closest to | 17 | 14 | 22 | 17 | 20 | 14 | 17 |
| knee) | | | | | | | |
| Graduated | Yes | Yes | No | Yes | Yes | Yes | 83.40% |

1. A conformable pressure sensor pad comprising:
a sensor arrangement including a central column of segments and outer columns of segments, wherein the outermost columns are tapered outwardly and wherein a pressure-sensitive sensor is mounted in a plurality of segments in the central column,
a conformable pad including a plurality of pouches, the pouches being in fluid communication with each other, wherein each segment of the sensor arrangement is positioned on a respective pouch of the conformable pad, the plurality of pouches being shaped to be positioned between a respective segment and a surface of a patient in use.

2. The conformable pressure sensor pad of claim 1, wherein the width of each respective pouch of the plurality of pouches is substantially equal to the width of the respective segment of the sensor arrangement.

3. The conformable pressure sensor pad of claim 1, wherein adjacent pouches are in fluid communication with each other via a fluid communication means located at the ends of adjacent pouches.

4. The conformable pressure sensor pad of claim 3, wherein a fluid communication means is located at alternative ends of each neighbouring pouch to create sets of interdigitated pouches.

5. The conformable pressure sensor pad of claim 1, wherein a fluid communication means is located at the edges of the pads to create a set of interdigitated pouches.

6. The conformable pressure sensor pad of claim 1, wherein a fluid communication means is an interdigitated section running centrally through the pad which creates sets of interdigitated pouches.

7. The conformable pressure sensor pad of claim 1, wherein the fluid communication means comprise pipes and/or openings.

8. The conformable pressure sensor pad of claim 1, wherein each pouch is a pouch of fluid.

9. The conformable pressure sensor pad of claim 1, wherein each pouch is a bellows structure with crenelated side walls

10. The conformable pressure sensor pad of claim 1, wherein a back surface of the segments of the sensor arrangement that is in contact with the conformable pad comprises an adhesive or high friction coefficient material.

11. The conformable pressure sensor pad of claim 1, wherein the conformable pad is pre-tensioned in a direction parallel to the circumference of the limb.

12. The conformable pressure sensor pad of claim 1, wherein each segment of the sensor arrangement extends along the length of a respective pouch.

13. The conformable pressure sensor pad of claim 12, wherein the length of each segment is substantially equal to the length of the respective pouch.

14. The conformable pressure sensor pad of claim 1 wherein the sensor arrangement includes an elongate member that is segmented, the segments being defined by continuous longitudinal and transverse grooves, such that adjacent segments are hinged together, wherein the longitudinal grooves define the central column of segments and the outer columns of segments.

15. The pressure sensor garment or bandage comprising a conformable pressure sensor pad according to claim 1 integrated therein.

16. The pressure sensor garment of claim 15, further comprising a plurality of conformable pressure sensor pads.

17. The pressure sensor garment of claim 15, wherein each conformable pressure sensor pad is positioned relative to one another to be interspersed along the length of a patient's limb onto which the pressure sensor garment is applied.

18.-19. (canceled)

20. The pressure sensor garment of claim 15, wherein the garment is configured to at least partly encircle a patient's limb.

21. The pressure sensor garment of claim 15, wherein the garment is one of a list comprising a sock, a wrap, a sleeve, and a band.

22. A conformable pressure sensor pad comprising:
a sensor strip comprising an elongate member that is segmented, the segments being defined by continuous longitudinal and transverse grooves, such that adjacent segments are hinged together, wherein the longitudinal grooves define a central column of segments and outer columns of segments, wherein the segments in the outermost columns are tapered outwardly, and wherein a pressure-sensitive sensor is mounted in a plurality of the segments in the central column; and
a conformable pad including a plurality of pouches, the pouches being in fluid communication with each other, each segment of the sensor strip being positioned on a respective pouch, the plurality of pouches being shaped to be positioned between a respective segment and a surface of a patient in use.
